(19) United States
(12) Patent Application Publication
(10) Pub. No.: US 2023/0329343 A1
(43) Pub. Date: Oct. 19, 2023

(54) VAPORIZER AND ELECTRONIC VAPORIZATION DEVICE

(71) Applicant: SHENZHEN SMOORE TECHNOLOGY LIMITED, Shenzhen (CN)

(72) Inventors: Hongming ZHOU, Shenzhen (CN); Hong LI, Shenzhen (CN); Huanxi LI, Shenzhen (CN); Ce PENG, Shenzhen (CN)

(21) Appl. No.: 18/339,048

(22) Filed: Jun. 21, 2023

Related U.S. Application Data
(63) Continuation of application No. PCT/CN2021/133814, filed on Nov. 29, 2021.

(30) Foreign Application Priority Data
Dec. 22, 2020 (CN) 202011525441.5

(51) Int. Cl. A24F 40/46; A24F 40/10; A24F 40/05; A24F 40/485; B05B 17/00; H05B 3/28

(52) U.S. Cl. CPC A24F 40/46; A24F 40/10; A24F 40/05; A24F 40/485; B05B 17/0646; H05B 3/28; H05B 2203/007; H05B 2203/021

(57) ABSTRACT

A vaporizer includes: an ultrasonic vaporization assembly having: a liquid inlet surface and a first support surface surrounding an edge of the liquid inlet surface, the first support surface and the liquid inlet surface being located on a same side of the ultrasonic vaporization assembly such that liquid enters the ultrasonic vaporization assembly from the liquid inlet surface to be vaporized to form liquid vapor; and a heating assembly attached to the first support for preheating liquid reaching near the liquid inlet surface.

### CROSS-REFERENCE TO PRIOR APPLICATION

This application is a continuation of International Patent Application No. PCT/CN2021/133814, filed on Nov. 29, 2021, which claims priority to Chinese Patent Application No. 202011525441.5, filed on Dec. 22, 2020. The entire disclosure of both applications is hereby incorporated by reference herein.

### FIELD

This application relates to the field of vaporization technologies, and in particular, to a vaporizer and an electronic vaporization device including the vaporizer.

### BACKGROUND

A vaporizer usually includes an ultrasonic vaporization piece, and a vaporization hole is provided in the ultrasonic vaporization piece. When vibrating at a high frequency, the ultrasonic vaporization piece may vaporize liquid in the vaporization hole to form liquid vapor, and the liquid vapor is sprayed from the vaporization hole to be absorbed by a user. However, liquid vapor generated by the conventional vaporizer irritates a respiratory tract of a user, which affects user experience.

### SUMMARY

In an embodiment, the present invention provides a vaporizer, comprising: an ultrasonic vaporization assembly, comprising: a liquid inlet surface and a first support surface surrounding an edge of the liquid inlet surface, the first support surface and the liquid inlet surface being located on a same side of the ultrasonic vaporization assembly such that liquid enters the ultrasonic vaporization assembly from the liquid inlet surface to be vaporized to form liquid vapor; and a heating assembly attached to the first support surface and configured to preheat liquid reaching near the liquid inlet surface.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a three-dimensional schematic structural diagram of a vaporizer according to an embodiment;

FIG. 2 is a three-dimensional schematic structural diagram of the vaporizer shown in FIG. 1 at another angle of view;

FIG. 3 is a schematic exploded structural view of the vaporizer shown in FIG. 1;

FIG. 4 is a planar schematic cross-sectional structural view of the vaporizer shown in FIG. 1;

FIG. 5 is a partial schematic structural diagram of FIG. 4;

FIG. 6 is an enlarged schematic structural diagram of A in FIG. 5; and

FIG. 7 is a planar schematic cross-sectional structural view of a vaporizer according to another embodiment.

### DETAILED DESCRIPTION

In an embodiment, the present invention provides a vaporizer and an electronic vaporization device including the vaporizer.

In an embodiment, the present invention provides a vaporizer, including:
an ultrasonic vaporization assembly, including a liquid inlet surface and a first support surface surrounding an edge of the liquid inlet surface, where the first support surface and the liquid inlet surface are located on a same side of the ultrasonic vaporization assembly, and liquid enters the ultrasonic vaporization assembly from the liquid inlet surface to be vaporized to form liquid vapor; and
a heating assembly, attached to the first support surface, and configured to preheat liquid reaching near the liquid inlet surface.

In one of the embodiments, the ultrasonic vaporization assembly includes a piezoelectric ceramic piece and a metal piece, the liquid inlet surface and the first support surface are both located on a first side of the metal piece, the metal piece further includes a second support surface corresponding to the first support surface and a vapor outlet surface corresponding to the liquid inlet surface on a second side opposite the first side, the piezoelectric ceramic piece is attached to the second support surface and is provided with a through hole corresponding to the vapor outlet surface, and the metal piece is provided with a vaporization hole penetrating the liquid inlet surface and the vapor outlet surface and in communication with the through hole.

In one of the embodiments, at least part of both the liquid inlet surface and the vapor outlet surface is a spherical surface, an opening of the spherical surface faces the first side, and the vaporization hole is provided on the spherical surface.

In one of the embodiments, the heating assembly includes a heating layer, the heating layer is attached to the first side of the metal piece, and the heating layer is configured to include a high temperature zone surrounding the liquid inlet surface and a low temperature zone surrounding the high temperature zone.

In one of the embodiments, a distance between the edge of the liquid inlet surface and the heating layer is less than a distance between an edge of the first support surface and the heating layer.

In one of the embodiments, the heating layer includes a first resistance wire surrounding the liquid inlet surface and a second resistance wire surrounding the first resistance wire, and a resistance of the first resistance wire is greater than a resistance of the second resistance wire.

In one of the embodiments, the heating assembly further includes a first insulating layer and a second insulating layer, the first insulating layer is attached to the first support surface, and the heating layer is sandwiched between the first insulating layer and the second insulating layer.

In one of the embodiments, a thermal conductivity of the second insulating layer is greater than a thermal conductivity of the first insulating layer.

In one of the embodiments, the heating assembly further includes a third insulating layer and a bonding layer, the heating layer is encapsulated in the third insulating layer, and the bonding layer is attached to the first support surface and connected to the third insulating layer.

In one of the embodiments, the heating assembly is configured to be enabled before the ultrasonic vaporization assembly.

In one of the embodiments, the vaporization hole is a conical hole, and a hole diameter of the vaporization hole is gradually reduced from the liquid inlet surface to the vapor outlet surface.

In one of the embodiments, the first insulating layer, the second insulating layer and the heating layer are provided with through holes in communication with each other, the through holes correspond to the liquid inlet surface, and the liquid passes through the through holes to be in contact with the liquid inlet surface and enter the vaporization hole to be vaporized.

In one of the embodiments, the thickness of the first insulating layer and/or the second insulating layer ranges from 5 μm to 20 μm.

In one of the embodiments, the thickness of the heating layer ranges from 5 μm to 40 μm. An electronic vaporization device is provided, including the vaporizer of any one of the above.

The heating assembly may preheat liquid to a temperature close to a body temperature, and when the ultrasonic vaporization assembly vaporizes the liquid to form liquid vapor, a temperature of the liquid vapor is close to the body temperature, so as to prevent the liquid vapor from irritating a respiratory tract of a human body and improve user experience. In addition, the heating assembly is directly attached to a back of the ultrasonic vaporization assembly, that is, the heating assembly is integrated and arranged on the ultrasonic vaporization assembly. Therefore, the heating assembly may heat liquid near the liquid inlet surface, i.e. a part of the liquid, so that heated liquid may directly enter the ultrasonic vaporization assembly for vaporization. In this way, the liquid is heated up quickly, energy consumption of the heating assembly is reduced, and time for waiting the liquid vapor to spray is reduced to improve the user experience. The heating assembly is directly arranged on the first support surface of the metal piece, that is, the heating assembly is located on a side on which the liquid inlet surface is located, so that the heating assembly can be in direct contact with the liquid, and heat generated by the heating assembly is directly transmitted to the liquid without an intermediate medium, thereby reducing heat loss, improving a heat utilization rate, making the liquid heat up quickly, and reducing time for waiting the liquid vapor to spray. In addition, there is no need to set an additional heat insulation part on the heating assembly, thus simplifying an overall structure of the vaporizer.

For ease of understanding this application, this application is described more comprehensively below with reference to the related accompanying drawings.

The accompanying drawings show exemplary implementations of this application. However, this application may be implemented in many different forms, and is not limited to the implementations described in this specification. On the contrary, the implementations are provided to make understanding of the disclosed content of this application more comprehensive.

It needs to be noted that, when a component is referred to as “being fixed to” another component, the component may be directly on the another component, or an intermediate component may be present. When a component is considered to be “connected to” another component, the component may be directly connected to the another component, or an intermediate component may also be present. The terms “inner”, “outer”, “left”, “right” and similar expressions used in this specification are only for purposes of illustration but not indicate a unique implementation.

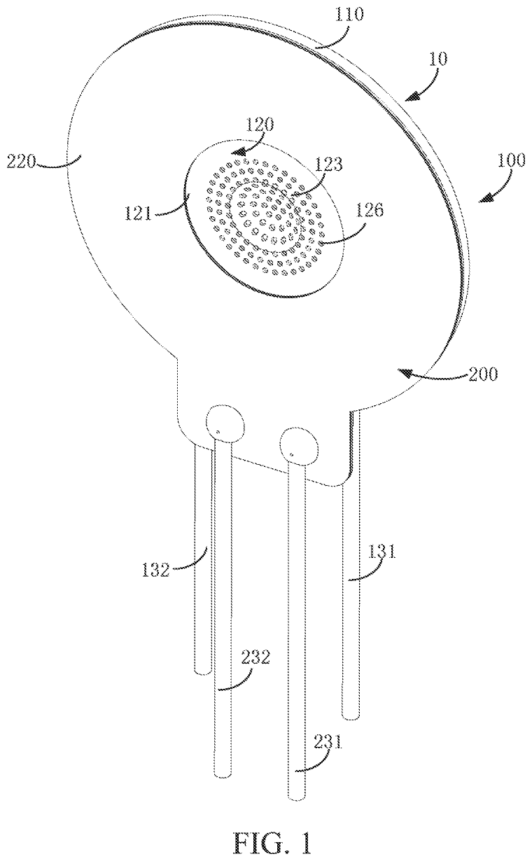

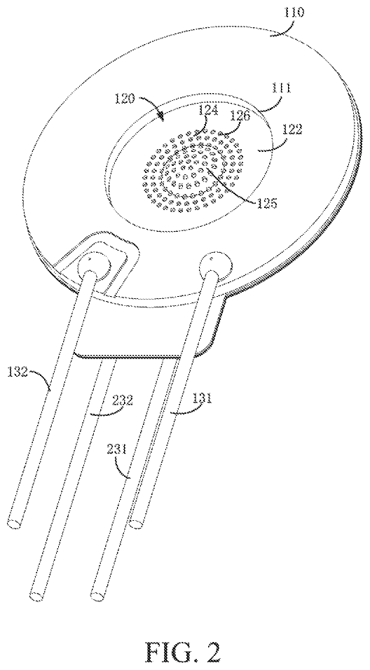

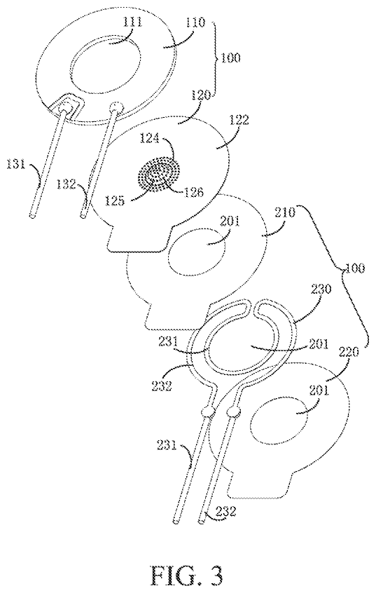

Referring to FIG. 1, FIG. 2, and FIG. 3, a vaporizer 10 provided in an embodiment of this application includes an ultrasonic vaporization assembly 100 and a heating assembly 200. The ultrasonic vaporization assembly 100 is configured to vaporize liquid to form liquid vapor, and the liquid may be oil, liquid medicine, or the like. When the liquid is vaporized to form the liquid vapor, a user may inhale the liquid vapor.

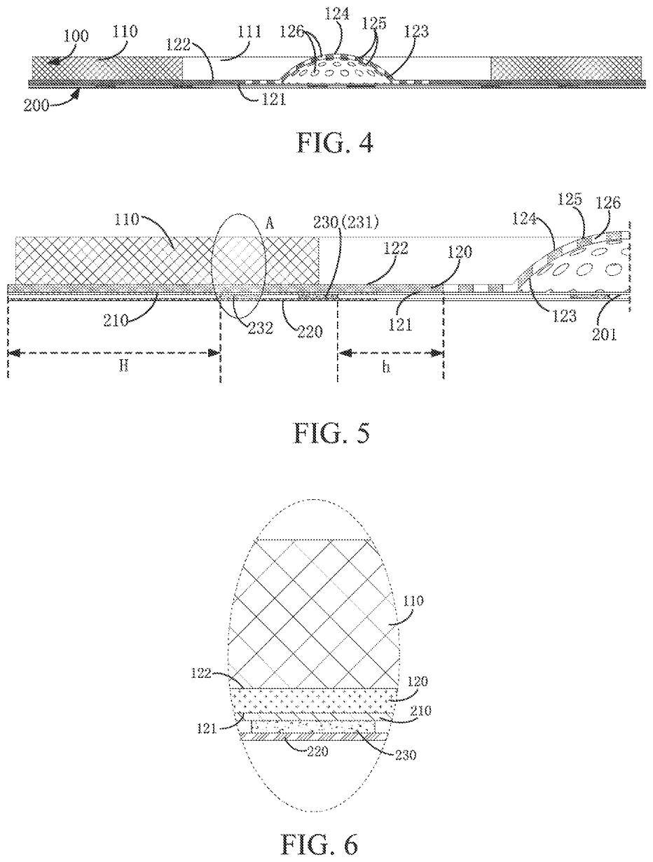

Referring to FIG. 3, FIG. 4, and FIG. 5, in some embodiments, the ultrasonic vaporization assembly 100 includes a piezoelectric ceramic piece 110, a metal piece 120, a first electrode 131, and a second electrode 132. An end of the first electrode 131 and an end of the second electrode 132 are electrically connected to a circuit in the piezoelectric ceramic piece 110, and another end of the first electrode 131 and another end of the second electrode 132 are configured to be connected to an alternating current power supply. Therefore, the circuit in the piezoelectric ceramic piece 110 is configured to pass an alternating current. When the alternating current power supply supplies the alternating current to the circuit in the piezoelectric ceramic piece 110 through the first electrode 131 and the second electrode 132, the piezoelectric ceramic piece 110 generates high frequency vibration, so that a vibration frequency of the piezoelectric ceramic piece 110 is equivalent to a vibration frequency of an ultrasound. A through hole 111 is provided at the center of the piezoelectric ceramic piece 110, and the through hole 111 penetrates two opposite surfaces of the piezoelectric ceramic piece 110. By providing the through hole 111, the piezoelectric ceramic piece 110 has a substantially circular structure.

The metal piece 120 has a substantially disk-shaped structure and includes a first support surface 121, a second support surface 122, a liquid inlet surface 123, and a vapor outlet surface 124. The liquid inlet surface 123 and the vapor outlet surface 124 are arranged in opposite surfaces and are located in central regions of the metal piece 120, and the second support surface 122 and the first support surface 121 are arranged in opposite surfaces and are located in edge regions of the metal piece 120. The second support surface 122 and the vapor outlet surface 124 are located on a second side (i.e. an upper side) of the metal piece 120, and the second support surface 122 is connected to a periphery of the vapor outlet surface 124, so that the second support surface 122 is circular and surrounds the vapor outlet surface 124. The first support surface 121 and the liquid inlet surface 123 are located on a first side (i.e. a lower side) of the metal piece 120, and the first support surface 121 is connected to a periphery of the liquid inlet surface 123, so that the first support surface 121 is circular and surrounds the liquid inlet surface 123. In other words, both the second support surface 122 and the first support surface 121 are spaced apart along a thickness direction of the metal piece 120 and correspond to each other, and both the liquid inlet surface 123 and the vapor outlet surface 124 are spaced apart along the thickness direction of the metal piece 120 and correspond to each other.

A plurality of vaporization holes 126 are provided on the metal piece 120, and the vaporization holes 126 penetrate both the liquid inlet surface 123 and the vapor outlet surface 124. The heating assembly 200 is arranged on the first support surface 121 of the metal piece 120. The piezoelectric ceramic piece 110 is attached to the second support surface 122 of the metal piece 120, so that the vapor outlet surface 124 corresponds to the through hole 111 of the piezoelectric ceramic piece 110, and the through hole 111 is in communication with the vaporization hole 126. Vaporized liquid is on a side at which the liquid inlet surface 123 is located, and the liquid is in direct contact with the liquid inlet surface 123, so that the liquid enters the vaporization hole 126 through the liquid inlet surface 123. When the piezoelectric ceramic piece 110 generates high frequency vibration under an action of an alternating current, vibration energy of the piezoelectric ceramic piece 110 is transmitted to the metal piece 120, so that the metal piece 120 also generates high frequency vibration following the piezoelectric ceramic piece 110, to vaporize the liquid in the vaporization hole 126 to form liquid vapor. The liquid vapor is sprayed from the vapor outlet surface 124 into the through hole 111 of the piezoelectric ceramic piece 110 to be absorbed by a user. In some embodiments, the vaporization hole 126 is a conical hole, and a hole diameter of the vaporization hole 126 is gradually reduced from the liquid inlet surface 123 to the vapor outlet surface 124.

The metal piece 120 may be made of a stainless steel material, so that the metal piece 120 has good structural strength, thermal conductivity, and rust resistance. Therefore, it may be ensured that the metal piece 120 has sufficient anti-fatigue strength, which prevents fatigue fracture of the metal piece 120 under high frequency vibration, and improves a service life of the entire ultrasonic vaporization assembly 100. In addition, it may prevent a rust particle from partially or completely blocking the vaporization hole 126, ensure that particle sizes of tiny liquid droplets in the liquid vapor are equal, and ensure that each vaporization hole 126 can vaporize the liquid, so as to finally improve uniformity and reliability of the metal piece 120 in vaporizing the liquid.

In some embodiments, at least a part of both the liquid inlet surface 123 and the vapor outlet surface 124 is a spherical surface 125. An opening of the spherical surface 125 is provided toward the heating assembly 200, so that an entire central region of the metal piece 120 forms a spherical protrusion, and an opening of the protrusion is the opening of the spherical surface 125.

The protrusion may also be formed by a central region of a flat metal piece 120 depressed toward the first side. Another part of the vaporization hole 126 may be located on the spherical surface 125, and another part of the vaporization hole 126 may be located at other parts of the liquid inlet surface 123 and the vapor outlet surface 124. Certainly, all vaporization holes 126 may be located on the spherical surface 125. Through configuration of the spherical surface 125, a plane perpendicular to a thickness direction of the metal piece 120 is used as a reference plane. Although an orthographic projection of a metal piece 120 having the spherical surface 125 on the reference plane is the same as that of the flat metal piece 120 on the reference plane, the metal piece 120 having the spherical surface 125 may ensure that the liquid vapor is sprayed in different directions and has a relatively large spraying range. In addition, a relatively large number of vaporization holes 126 may be provided, so that a vaporization amount of liquid per unit time is increased to increase liquid vapor concentration.

Referring to FIG. 3, FIG. 5, and FIG. 6, in some embodiments, the heating assembly 200 includes a first insulating layer 210, a second insulating layer 220, and a heating layer 230. The first insulating layer 210, the second insulating layer 220, and the heating layer 230 are all approximately circular, and are provided with through holes 201 in communication with each other. The through holes 201 correspond to the liquid inlet surface 123 of the metal piece 120, and liquid may pass through the through holes 201 to be in contact with the liquid inlet surface 123 and enter the vaporization hole 126 to be vaporized. The first insulating layer 210 is attached to the first support surface 121 of the metal piece 120, and the first insulating layer 210 may be attached to the first support surface 121 through a physical vapor deposition (PVD) process or a screen printing process, so that the first insulating layer 210 is directly connected to the metal piece 120, to prevent the first insulating layer 210 from being connected to the metal piece 120 through another connection layer, and reduce a thickness and weight of the entire vaporizer 10, which facilitates a thin and light design of the vaporizer 10. The thickness of the first insulating layer 210 may range from 5 μm to 20 μm, for example, a specific value may be 5 μm, 10 μm, 15 μm, 20 μm, or the like. On the basis of ensuring that the first insulating layer 210 has sufficient insulating performance, the thickness of the first insulating layer 210 may be appropriately reduced, so that the thickness of the entire vaporizer 10 may be further compressed.

The heating layer 230 is attached to a surface of the first insulating layer 210 facing away from the metal piece 120, and the heating layer 230 may also be attached to the first insulating layer 210 through the physical vapor deposition (PVD) process or the screen printing process, so that the heating layer 230 is directly connected to the first insulating layer 210, to prevent the heating layer 230 from being connected to the first insulating layer 210 through another connecting layer, which may also reduce the thickness of the entire vaporizer 10 and realize the thin and light design of the vaporizer 10. The thickness of the heating layer 230 may range from 5 μm to 40 μm, for example, a specific value may be 5 μm, 20 μm, 30 μm, 40 μm, or the like. The heating layer 230 may further include a third electrode 231 and a fourth electrode 232. An end of the third electrode 231 and an end of the fourth electrode 232 are electrically connected to the heating layer 230, and another end of the third electrode 231 and another end of the fourth electrode 232 are configured to be connected to a direct current power supply. Therefore, the heating layer 230 is configured to pass a direct current. The heating layer 230 may convert electrical energy to heat energy when the direct current power supply (e.g. a battery) applies a direct current to the heating layer 230 through the third electrode 231 and the fourth electrode 232.

The heating layer 230 may be a layered structure formed by bending a wire-like wire or directly be a layered structure formed by a conductive diaphragm. The heating layer 230 may be a carbon nanoflake or may be made of a metal or alloy material such as a stainless steel material, a titanium metal material, or a titanium alloy material.

The second insulating layer 220 is attached to a surface of the heating layer 230 facing away from the metal piece 120, and the second insulating layer 220 may also be attached to the heating layer 230 through the physical vapor deposition (PVD) process or the screen printing process, so that the heating layer 230 is directly connected to the second insulating layer 220, to prevent the heating layer 230 from being connected to the second insulating layer 220 through another connecting layer, which may also reduce the thickness of the entire vaporizer 10 and realize the thin and light design of the vaporizer 10. The thickness of the second insulating layer 220 may range from 5 μm to 20 μm, for example, a specific value may be 5 μm, 10 μm, 15 μm, 20 μm, or the like. On the basis of ensuring that the second insulating layer 220 has sufficient insulating performance, the thickness of the second insulating layer 220 may be appropriately reduced, so that the thickness of the entire vaporizer 10 may be further compressed. The second insulating layer 220 may be made of a ceramic glaze material, so that the second insulating layer 220 has high wear resistance and good thermal conductivity.

Therefore, for the entire heating assembly 200, the first insulating layer 210 is arranged on the first support surface 121 of the metal piece 120, and the heating layer 230 is directly sandwiched between the first insulating layer 210 and the second insulating layer 220. A short circuit phenomenon caused by a direct contact between the heating layer 230 and the metal piece 120 may be avoided through arrangement of the first insulating layer 210. Through arrangement of the second insulating layer 220, the second insulating layer 220 is in direct contact with liquid on a side of the liquid inlet surface 123, so as to avoid a short circuit phenomenon caused by a direct contact between the heating layer 230 and the liquid, and also avoid contamination on the liquid caused by a contact between the heating layer 230 and the liquid.

The heating assembly 200 is enabled before the ultrasonic vaporization assembly 100 in a whole working process of the vaporizer 10. Specifically, a direct current power supply is used for supplying power to the heating layer 230 through the third electrode 231 and the fourth electrode 232, and then an alternating current power supply is used for supplying power to the circuit in the piezoelectric ceramic piece 110 through the first electrode 131 and the second electrode 132, so that a working time of the heating layer 230 is earlier than a working time of the piezoelectric ceramic piece 110. For example, the heating layer 230 may be enabled no more than one second before the piezoelectric ceramic piece 110, so that the piezoelectric ceramic piece 110 may drive the metal piece 120 to vibrate to vaporize liquid to form liquid vapor as soon as possible, which reduces time of waiting for the liquid vapor by a user, and improves user experience of the entire vaporizer 10.

By first enabling the heating assembly 200, the heating assembly 200 may preheat liquid reaching near the liquid inlet surface 123. Certainly, the heating assembly 200 may preheat the liquid to a temperature close to a body temperature. When the metal piece 120 vaporizes the liquid to form liquid vapor sprayed from the vapor outlet surface 124 into the through hole 111, the temperature of the liquid vapor is close to the body temperature. Especially for vaporization of liquid medicine, when the user absorbs the liquid medicine whose temperature is close to the body temperature and which exists in a form of liquid vapor, the liquid medicine (liquid vapor) may be prevented from irritating a respiratory tract of a human body, so as to avoid other symptoms such as cough or elevated blood pressure, and ensure a therapeutic effect of the liquid medicine on the user. In addition, a temperature difference between the liquid vapor in an early stage and a middle-late stage of the heating assembly 200 due to simultaneous enabling of the heating assembly 200 and the piezoelectric ceramic piece 110 may be eliminated, the temperature of the liquid vapor in a vaporization process may be kept consistent, and comfort of inhaling the liquid vapor may be improved. Further, for liquid with relatively high viscosity, through a preheating action of the heating assembly 200, the viscosity of the liquid may be appropriately reduced, thereby increasing fluidity of the liquid, making it easier for the liquid to enter the vaporization hole 126 and to be rapidly vaporized to form liquid vapor under high frequency vibration, avoiding a phenomenon that the liquid is difficult to flow into the vaporization hole 126 or is difficult to be vaporized to form the liquid vapor, and improving vaporization efficiency, vaporization stability, and vaporization reliability of the entire vaporizer 10.

If a separate heating assembly 200 is used for heating the liquid, the heating assembly 200 simultaneously heat liquid near the liquid inlet surface 123 and liquid far away from the liquid inlet surface 123. When the liquid far away from the liquid inlet surface 123 reaches near the liquid inlet surface 123, the heating assembly 200 repeatedly heats the liquid, resulting in a waste of energy. In addition, due to a large amount of heated liquid, the liquid is heated up slowly, which also prolongs waiting time of the user. In the foregoing embodiment, the heating assembly 200 is directly attached to the first support surface 121 of the ultrasonic vaporization assembly 100, that is, the heating assembly 200 is integrated and arranged on the ultrasonic vaporization assembly 100. Therefore, the heating assembly 200 may merely heat liquid near the liquid inlet surface 123, i.e. a part of the liquid, so that heated liquid may directly enter the vaporization hole 126 for vaporization. In this way, the liquid is heated up quickly, energy consumption of the heating assembly 200 is reduced, and time for waiting the liquid vapor to spray is reduced to improve the user experience.

If the heating assembly 200 is arranged on a side on which the vapor outlet surface 124 of the metal piece 120 is located, the heating assembly 200 cannot be in direct contact with liquid on a side of the liquid inlet surface 123, and heat generated by the heating assembly 200 is transferred to the liquid through the metal piece 120 to preheat the liquid, thus prolonging a heat transfer path, increasing a heat loss generated in a heat transfer process, and reducing a heat utilization rate of the heating assembly 200. In addition, the heating assembly 200 needs to be protected by a heat insulation part, to prevent heat of the heating assembly 200 from being transferred to another member of the vaporizer 10 and causing damage to the member. Therefore, arrangement of the heat insulation part significantly complicates a structure of the vaporizer 10. However, in the foregoing embodiment, the entire heating assembly 200 is directly arranged on the first support surface 121 of the metal piece 120, that is, the heating assembly 200 is located on a side on which the liquid inlet surface 123 is located, so that the heating assembly 200 can be in direct contact with the liquid, and heat generated by the heating assembly 200 is directly transmitted to the liquid without an intermediate medium, thereby reducing heat loss, improving a heat utilization rate, making the liquid heat up quickly, and reducing time for waiting the liquid vapor to spray. In addition, there is no need to set an additional heat insulation part on the heating assembly 200, thus simplifying an overall structure of the vaporizer 10. In some embodiments, the heating layer 230 is configured to include a high temperature zone surrounding the liquid inlet surface 123 and a low temperature zone surrounding the high temperature zone, so that the high temperature zone of the heating layer 230 may be closer to the liquid inlet surface 123 than the low temperature zone. In some embodiments, the heating layer 123 includes a first resistance wire 231 surrounding the liquid inlet surface 123 and a second resistance wire 232 surrounding the first resistance wire. A resistance of the first resistance wire 231 is greater than a resistance of the second resistance wire 232. Because the first resistance wire 231 and the second resistance wire 232 are connected in series, heat generated by the first resistance wire 231 is more than heat generated by the second resistance wire 232 in a same time period, so that the first resistance wire 231 corresponds to the high temperature zone and the second resistance wire 232 corresponds to the low temperature zone. Therefore, heat generated by the heating layer 230 is more focused on the liquid near the liquid inlet surface 123, which may reduce repeated heating and increase a heating speed of the liquid. In addition, heat transferred by the heating layer 230 to another member of the vaporizer 10 through an edge of the metal piece 120 may be reduced and an energy utilization rate of the heating layer 230 may be improved. In other embodiments, in a case that the resistance of the first resistance wire 231 is equal to the resistance of the second resistance wire 232, the liquid inlet surface 123 may be closer to the heating layer 230 relative to an edge of the first support surface 121 of the metal piece 120. In other words, in a case that a distance h between the heating layer 230 and the liquid inlet surface 123 is smaller than a distance H between the heating layer 230 and the edge of the first support surface 121, the heat generated by the heating layer 230 may also be more focused on the liquid near the liquid inlet surface 123, thus improving the energy utilization rate of the heating layer 230. A thermal conductivity of the second insulating layer 220 is greater than a thermal conductivity of the first insulating layer 210, so that the heat generated by the heating layer 230 may be easily and directly transferred to the liquid through the second insulating layer 220, heat transferred to another member of the vaporizer 10 through the first insulating layer 210 and the metal piece 120 may be reduced, and the energy utilization rate of the heating layer 230 may also be improved.

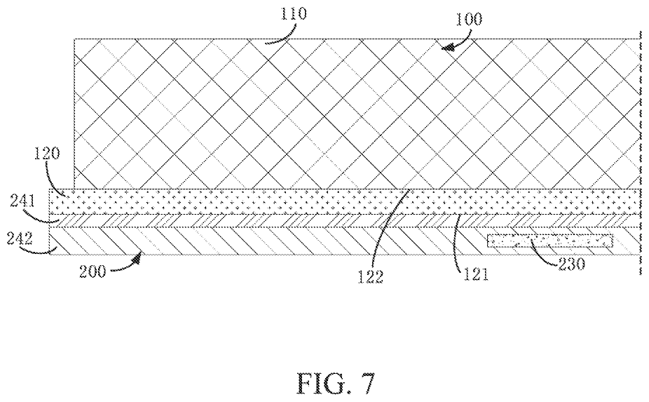

Referring to FIG. 7, in some embodiments, during a manufacturing process of the heating assembly 200, for example, a prepared heating layer 230 may be placed in an injection mold and then a liquid insulating material is injected to a cavity of the mold. The liquid insulating material may be cooled and solidified to form an insulating layer, and the insulating layer may be denoted as a third insulating layer 241. Therefore, the entire heating layer 230 is encapsulated in the third insulating layer 241, and short circuit caused by a contact between the heating layer 230, and the metal piece 120 as well as the liquid is also avoided through an action of the third insulating layer 241. In some embodiments, the third insulating layer 241 may be provided with a slot structure, so that a third electrode 231 and a fourth electrode 232 electrically connected to the heating layer 230 penetrate the slot structure.

During a mounting process of the heating assembly 200, a bonding layer 242 may be arranged on the first support surface 121 of the metal piece 120, and the third insulating layer 241 is attached to the bonding layer 242. That is, the third insulating layer 241 is arranged on the first support surface 121 of the metal piece 120 through the bonding layer 242. Certainly, the third insulating layer 241 may be fixed on the first support surface 121 of the metal piece 120 through a snap connection.

In another example, an insulating layer may be arranged on both sides of the prepared heating layer 230 through physical vapor deposition, electrophoresis, or spraying, and then the insulating layer is fixed on the metal piece 120 through the bonding layer 242 or the snap connection.

This application further provides an electronic vaporization device. The electronic vaporization device includes the vaporizer 10. By arranging the vaporizer 10, user experience of the electronic vaporization device may be improved, energy consumption may be reduced, and a structure may be thinner and lighter.

The technical features in the foregoing embodiments may be randomly combined. For concise description, not all possible combinations of the technical features in the embodiment are described. However, provided that combinations of the technical features do not conflict with each other, the combinations of the technical features are considered as falling within the scope recorded in this specification.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A vaporizer, comprising:
an ultrasonic vaporization assembly, comprising:
a liquid inlet surface and a first support surface surrounding an edge of the liquid inlet surface, the first support surface and the liquid inlet surface being located on a same side of the ultrasonic vaporization assembly such that liquid enters the ultrasonic vaporization assembly from the liquid inlet surface to be vaporized to form liquid vapor; and
a heating assembly attached to the first support surface and configured to preheat liquid reaching near the liquid inlet surface.

2. The vaporizer of claim 1, wherein the ultrasonic vaporization assembly comprises a piezoelectric ceramic piece and a metal piece,
wherein the liquid inlet surface and the first support surface are both located on a first side of the metal piece,
wherein the metal piece comprises a second support surface corresponding to the first support surface and a vapor outlet surface corresponding to the liquid inlet surface on a second side opposite the first side,
wherein the piezoelectric ceramic piece is attached to the second support surface and is provided with a through hole corresponding to the vapor outlet surface, and
wherein the metal piece is provided with a vaporization hole penetrating the liquid inlet surface and the vapor outlet surface and in communication with the through hole.

3. The vaporizer of claim 2, wherein at least part of both the liquid inlet surface and the vapor outlet surface is a spherical surface,
wherein an opening of the spherical surface faces the first side, and
wherein the vaporization hole is provided on the spherical surface.

4. The vaporizer of claim 2, wherein the heating assembly comprises a heating layer attached to a first side of the metal piece, the heating layer comprising a high temperature zone surrounding the liquid inlet surface and a low temperature zone surrounding the high temperature zone.

5. The vaporizer of claim 4, wherein a distance between the edge of the liquid inlet surface and the heating layer is less than a distance between an edge of the first support surface and the heating layer.

6. The vaporizer of claim 4, wherein the heating layer comprises a first resistance wire surrounding the liquid inlet surface and a second resistance wire surrounding the first resistance wire, a resistance of the first resistance wire being greater than a resistance of the second resistance wire.

7. The vaporizer of claim 4, wherein the heating assembly further comprises a first insulating layer and a second insulating layer,
wherein the first insulating layer is attached to the first support surface, and the heating layer is sandwiched between the first insulating layer and the second insulating layer.

8. The vaporizer of claim 7, wherein a thermal conductivity of the second insulating layer is greater than a thermal conductivity of the first insulating layer.

9. The vaporizer of claim 4, wherein the heating assembly further comprises a third insulating layer and a bonding layer, the heating layer being encapsulated in the third insulating layer, and
wherein the bonding layer is attached to the first support surface and connected to the third insulating layer.

10. The vaporizer of claim 1, wherein the heating assembly is configured to be enabled before the ultrasonic vaporization assembly.

11. The vaporizer of claim 2, wherein the vaporization hole is a conical hole, and a hole diameter of the vaporization hole is gradually reduced from the liquid inlet surface to the vapor outlet surface.

12. The vaporizer of claim 7, wherein the first insulating layer, the second insulating layer, and the heating layer are provided with through holes in communication with each other, the through holes corresponding to the liquid inlet surface, such that the liquid passes through the through holes to be in contact with the liquid inlet surface and enter the vaporization hole to be vaporized.

13. The vaporizer of claim 7, wherein a thickness of the first insulating layer and/or the second insulating layer ranges from 5 μm to 20 μm.

14. The vaporizer of claim 4, wherein a thickness of the heating layer ranges from 5 μm to 40 μm.

15. An electronic vaporization device, comprising:
the vaporizer of claim 1.
